SEQUENCE LISTING (39 sequences)

SEQ ID NO: 1 - protein, 16 aa, unidentified
RQIKIWFQNRRMKWKK

SEQ ID NO: 2 - protein, 9 aa, unidentified
RKKRRQRRR

SEQ ID NO: 3 - protein, 17 aa, unidentified
TRQARRNRRRRWRERQR

SEQ ID NO: 4 - protein, 9 aa, unidentified
RRRRRRRRR

SEQ ID NO: 5 - protein, 8 aa, unidentified
RRRRRRRR

SEQ ID NO: 6 - DNA, 18 bp, Homo sapiens
  source 1..18: mol_type = other DNA
ccaaccgcgagaagatga

SEQ ID NO: 7 - DNA, 20 bp, Homo sapiens
  source 1..20: mol_type = other DNA
ccagaggcgtacagggatag

SEQ ID NO: 8 - DNA, 18 bp, Homo sapiens
  source 1..18: mol_type = other DNA
gacggacccgtcactctc

SEQ ID NO: 9 - DNA, 18 bp, Homo sapiens
  source 1..18: mol_type = other DNA
gtgcccgtggtcaattct

SEQ ID NO: 10 - DNA, 21 bp, Homo sapiens
  source 1..21: mol_type = other DNA
caccgaccttgcaaatactga

SEQ ID NO: 11 - DNA, 21 bp, Homo sapiens
  source 1..21: mol_type = other DNA
tgggaggtacaactttaacca

SEQ ID NO: 12 - DNA, 24 bp, Homo sapiens
  source 1..24: mol_type = other DNA
gcatttaaaccttttactgctgtg

SEQ ID NO: 13 - DNA, 27 bp, Homo sapiens
  source 1..27: mol_type = other DNA
tcagcatgaaagagaatattacagaaa

SEQ ID NO: 14 - DNA, 21 bp, Homo sapiens
  source 1..21: mol_type = other DNA
aagggacccaagagatgaaga

SEQ ID NO: 15 - DNA, 20 bp, Homo sapiens
  source 1..20: mol_type = other DNA
cctttgattgccacaattcc

SEQ ID NO: 16 - DNA, 20 bp, Homo sapiens
  source 1..20: mol_type = other DNA
gcaaagccgacctatgctac

SEQ ID NO: 17 - DNA, 20 bp, Homo sapiens
  source 1..20: mol_type = other DNA
gagcctctagtgcctggaca

SEQ ID NO: 18 - DNA, 18 bp, Homo sapiens
  source 1..18: mol_type = other DNA
cctccgtgcacctagcag

SEQ ID NO: 19 - DNA, 18 bp, Homo sapiens
  source 1..18: mol_type = other DNA
cagctcaagctcggatgg

SEQ ID NO: 20 - DNA, 21 bp, Homo sapiens
  source 1..21: mol_type = other DNA
cagagggagagggagagagag

SEQ ID NO: 21 - DNA, 20 bp, Homo sapiens
  source 1..20: mol_type = other DNA
tccagaacatcctgcaatga

SEQ ID NO: 22 - DNA, 20 bp, Homo sapiens
  source 1..20: mol_type = other DNA
cagttcatgtcccgcgccaa

SEQ ID NO: 23 - DNA, 20 bp, Homo sapiens
  source 1..20: mol_type = other DNA
gcctcgaagaccaccgtcac

SEQ ID NO: 24 - DNA, 22 bp, Homo sapiens
  source 1..22: mol_type = other DNA
cacacagggttgtacacttgct

SEQ ID NO: 25 - DNA, 20 bp, Homo sapiens
  source 1..20: mol_type = other DNA
aaggctacatctgggtctgg

SEQ ID NO: 26 - DNA, 20 bp, Homo sapiens
  source 1..20: mol_type = other DNA
gcctgtaggaagtggcttgt

SEQ ID NO: 27 - DNA, 20 bp, Homo sapiens
  source 1..20: mol_type = other DNA
gaggtgaggggaaaccattt

SEQ ID NO: 28 - DNA, 24 bp, Homo sapiens
  source 1..24: mol_type = other DNA
tgaaattttgattcagacatccat

SEQ ID NO: 29 - DNA, 20 bp, Homo sapiens
  source 1..20: mol_type = other DNA
tttgggtttatgggttcagg

SEQ ID NO: 30 - DNA, 18 bp, Homo sapiens
  source 1..18: mol_type = other DNA
tttggcccaccagaacac

SEQ ID NO: 31 - DNA, 18 bp, Homo sapiens
  source 1..18: mol_type = other DNA
ggtccttcctgcccttct

SEQ ID NO: 32 - DNA, 21 bp, Homo sapiens
  source 1..21: mol_type = other DNA
caacaatgagctttcccattt

SEQ ID NO: 33 - DNA, 21 bp, Homo sapiens
  source 1..21: mol_type = other DNA
cattgtccagtgtttccatga

SEQ ID NO: 34 - DNA, 23 bp, Homo sapiens
  source 1..23: mol_type = other DNA
gtatgtttggttccatgtcacag

SEQ ID NO: 35 - DNA, 18 bp, Homo sapiens
  source 1..18: mol_type = other DNA
ggacacgtgggaactcca

SEQ ID NO: 36 - DNA, 20 bp, Homo sapiens
  source 1..20: mol_type = other DNA
tctgcagtgtcccatgaagt

SEQ ID NO: 37 - DNA, 20 bp, Homo sapiens
  source 1..20: mol_type = other DNA
cagcatcagggtaggtagca

SEQ ID NO: 38 - DNA, 18 bp, Homo sapiens
  source 1..18: mol_type = other DNA
catgtccgcctcccacta

SEQ ID NO: 39 - DNA, 18 bp, Homo sapiens
  source 1..18: mol_type = other DNA
cgaatgcatcccaattcc

(19) United States
(12) Patent Application Publication
(10) Pub. No.: US 2025/0002848 A1
(43) Pub. Date: Jan. 2, 2025

(54) TRANSDIFFERENTIATION OF NON-DERMAL PAPILLA CELLS TO DERMAL PAPILLA CELLS

(71) Applicant: dNovo Inc., San Jose, CA (US)

(72) Inventors: Ernesto Lujan, San Jose, CA (US); Aaron Coey, San Jose, CA (US)

(21) Appl. No.: 18/688,299

(22) PCT Filed: Oct. 20, 2022
(86) PCT No.: PCT/US2022/078422

§ 371 Date: Feb. 29, 2024

Related U.S. Application Data
(60) Provisional application No. 63/257,752, filed on Oct. 20, 2021.

(51) Int. Cl. C12N 5/071; C12N 15/86

(52) U.S. Cl. CPC C12N 5/0625; C12N 15/86; C12N 2506/09; C12N 2510/00; C12N 2740/15043

(57) ABSTRACT

Provided are compositions of a system and methods of use thereof to transdifferentiate non-dermal papilla (DP) cells into induced dermal papilla cells (iDPCs) and to subsequent use of these cells in the treatment of hair loss.

### CROSS-REFERENCE TO RELATED APPLICATIONS

This application claims the benefit of and priority to U.S. Provisional Patent Application No. 63/257,752, filed Oct. 20, 2021, the disclosure of which is hereby incorporated by reference in its entirety for all purposes.

### SEQUENCE LISTING

The instant application contains a Sequence Listing which has been submitted electronically in ST.26 format and is hereby incorporated by reference in its entirety. Said ST.26 copy, created on Sep. 29, 2022, is named DNO-002WO.xml and is 34,787 bytes in size.

### FIELD OF THE INVENTION

This invention generally relates to compositions for transdifferentiation of non-dermal papilla cells to dermal papilla (DP) cells for use in treating hair loss.

### BACKGROUND

Effective therapies to treat hair loss (otherwise known as alopecia) have been extensively researched for decades, but currently available treatments are insufficient for long term reversal of the condition. Pharmaceutical therapies can be used to treat underlying conditions that drive hair loss, such as the use of Minoxidil (Rogaine) or Finasteride (Propecia) for hereditary baldness; however, many patients see little, if any, benefit from these therapies, and those patients that do benefit from pharmaceutical intervention must remain on the medication indefinitely to maintain therapeutic effects.

Hair transplantation surgery is an alternative to pharmaceutical intervention. With this procedure, patient hair follicles are taken from a donor site and re-grafted to another part of the scalp as an autograft. Unfortunately, this procedure is only possible in patients exhibiting early stages of hair loss as a sufficient amount of patient donor sites are necessary to perform the procedure. As hereditary hair loss progresses, these donor hair sites become smaller in area and number making successful transplantation less probable. Additionally, hair transplantation surgery may be disappointing as it leads to a decrease in hair density at the donor site, and hair density across the entire scalp will never reach pre-hair loss density due to a lack of donor hair follicles. More recently, laser therapy has been approved for the treatment of hereditary hair loss, however the long-term effects of this therapy are unclear. As a consequence, new therapeutic strategies are needed to promote long-term hair growth that do not require indefinite medication or rely on a finite supply of donor hair follicles.

A promising therapeutic strategy may be the use of stem cells to repopulate cells within hair follicles. Similar to how bone marrow transplantation may be used to reverse hematopoietic disease, dermal papillae (DPs) may be used to reverse hair loss. This therapy would provide the benefits of hair transplants without the need to rely on a finite source of patient donor hair follicles by using cells that may self-renew and provide long-term reversal of hair loss.

A major challenge to the use of DPs is the ability to obtain an effective amount to use in therapy. DPs are low prevalence and must be harvested from existing hair follicles. Furthermore, there are no known methods of transdifferentiating non-dermal papilla (non-DP) cells into induced dermal papilla cells (iDPCs). Therefore, any method of efficiently generating iDPCs from non-DP cells and effectively grafting them into patient skin would present a promising step in the treatment of hair loss.

### BRIEF SUMMARY OF THE INVENTION

The present disclosure is directed to compositions of a system and methods of use thereof to transdifferentiate non-dermal papilla (non-DP) cells into induced dermal papilla cells (iDPCs) and to subsequent use of these cells in the treatment of hair loss.

Provided herein is a composition for transdifferentiation comprising a population of non-DP cells; a population of DPs expressing one or more marker(s) selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2; and a dermal papilla cell reprogramming (DPCR) system comprising one or more DPCR factor(s) selected from the group consisting of an EBF agent and a TWIST agent, wherein the DPCR system causes transdifferentiation of one or more non-DP cell into one or more iDPC.

In some embodiments, the population of iDPCs have at least a two-fold increased expression of one or more marker(s), selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2, as compared to the population of non-DP cells. In some embodiments, the population of iDPCs have at least a five-fold increased expression of one or more marker(s), selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2, as compared to the population of non-DP cells. In some embodiments, the population of iDPCs have at least a ten-fold increased expression of one or more marker(s), selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2, as compared to the population of non-DP cells. In some embodiments, expression of the one or more marker(s) is maintained over at least one cell passage. In some embodiments, expression of the one or more marker(s) is determined by messenger RNA analysis using a method selected from the group consisting of quantitative polymerase chain reaction (PCR), reverse transcription PCR, RNA-Seq, or northern blot. In some embodiments, expression of the one or more marker(s) is determined by protein analysis using a method selected from the group consisting of flow cytometry, or western blot.

In some embodiments, the EBF agent is an EBF polypeptide, or functional fragment thereof, fused to a permeant domain. In some embodiments, the EBF agent is a nucleic acid encoding an EBF polypeptide, or functional fragment thereof. In some embodiments, the TWIST agent is a TWIST polypeptide, or functional fragment thereof, fused to a permeant domain. In some embodiments, the TWIST agent is a nucleic acid encoding a TWIST polypeptide, or functional fragment thereof. In some embodiments, the nucleic acids comprise a tetracycline-responsive promoter. In some embodiments, the nucleic acid is within a capsid of a viral particle. In some embodiments, the permeant domain comprises an amino acid sequence selected from the group consisting of:

(SEQ ID NO: 1)
RQIKIWFQNRRMKWKK,
(amino acids 49-57 of HIV-1 tat; SEQ ID NO: 2)
RKKRRQRRR,
(amino acids 34-50 of HIV-1 rev; SEQ ID NO: 3)
TRQARRNRRRRWRERQR,
(R9; SEQ ID NO: 4)
RRRRRRRRR,
and
(R8; SEQ ID NO: 5)
RRRRRRRR.

In some embodiments, the population of non-DP cells are mammalian cells. In some embodiments, the population of non-DP cells are human or murine cells. In some embodiments, the non-DP cells are selected from the group consisting of dermal cells, epidermal cells, epithelial cells, adipocytes, and hematopoietic cells. In some embodiments the dermal cells are selected from the group consisting of fibroblasts, smooth muscle cells, and connective tissue cells.

Also provided herein is a method of making a population of iDPCs, comprising obtaining a population of non-DP cells; contacting the population of non-DP cells with a DPCR system comprising one or more DPCR factor(s) selected from the group consisting of an EBF agent and a TWIST agent; and incubating the population of non-DP cells in the presence of the DPCR system, wherein one or more non-DP cell is transdifferentiated into one or more iDPC.

In some embodiments of a method provided herein, the non-DP cell samples are collected from a mammalian subject, for example, a human or murine subject. In some embodiments, the non-DP cells are selected from the group consisting of dermal cells, epidermal cells, epithelial cells, adipocytes, and hematopoietic cells. In some embodiments the dermal cells are selected from the group consisting of fibroblasts, smooth muscle cells, and connective tissue cells. In some embodiments, the population of non-DP cells are incubated in the presence of the DPCR system is for a period of 24 hours to 6 weeks. In some embodiments, the non-DP cell samples are collected from a subject to whom the transdifferentiated one or more iDPC is administered. In some embodiments, the non-DP cells are collected from a subject who is different than the subject to whom the transdifferentiated one or more iDPC is administered.

In some embodiments of a method provided herein, the one or more iDPC expresses one or more marker(s) selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2. In some embodiments, the population of iDPCs have at least a two-fold increased expression of one or more marker(s) selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2, as compared to the population of non-DP cells. In some embodiments, the population of iDPCs have at least a five-fold increased expression of one or more marker(s) selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2, as compared to the population of non-DP cells. In some embodiments, the population of iDPCs have at least a ten-fold increased expression of one or more marker(s) selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2, as compared to the population of non-DP cells.

In some embodiments, the method comprises passaging the iDPCs at least once. In some embodiments, expression of the one or more marker(s) is maintained over at least one cell passage.

In some embodiments of a method provided herein, the EBF agent is an EBF polypeptide, or functional fragment thereof, fused to a permeant domain. In some embodiments, the EBF agent is a nucleic acid encoding an EBF polypeptide, or functional fragment thereof. In some embodiments, the TWIST agent is a TWIST polypeptide, or functional fragment thereof, fused to a permeant domain. In some embodiments, the TWIST agent is a nucleic acid encoding an TWIST polypeptide, or functional fragment thereof. In some embodiments, the nucleic acids comprise a tetracycline-responsive promoter. In some embodiments, the nucleic acid is within a capsid of a viral particle. In some embodiments, the permeant domain comprises an amino acid sequence selected from the group consisting of:

(SEQ ID NO: 1)
RQIKIWFQNRRMKWKK,
(amino acids 49-57 of HIV-1 tat; SEQ ID NO: 2)
RKKRRQRRR,
(amino acids 34-50 of HIV-1 rev; SEQ ID NO: 3)
TRQARRNRRRRWRERQR,
(R9; SEQ ID NO: 4)
RRRRRRRRR,
and
(R8; SEQ ID NO: 5)
RRRRRRRR.

In some embodiments of a method provided herein, the population of iDPCs are selected and enriched based on the expression of the one or more marker(s) selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2. In some embodiments, selecting and enriching comprises fluorescence-activated cell sorting (FACS) or magnetic-activated cell sorting (MACS). In some embodiments, selecting and enriching comprises dissociating the population of iDPCs to form a cell suspension and adding a population of dermal cells to the cell suspension. In some embodiments the dermal cells are selected from the group consisting of fibroblasts, smooth muscle cells, connective tissue cells, and adipocytes.

In some embodiments of a method provided herein, the population of iDPCs are dissociated to form a cell suspension and a population of epidermal cells is added to the cell suspension.

In some embodiments of a method provided herein, the iDPCs are cultured on a scaffold. In some embodiments, the scaffold is composed of a plastic, Matrigel or a similar basement membrane extract, gelatin, collagen, or laminin. In some embodiments the iDPCs are cultured on the scaffold for 30 minutes to 48 hours.

In some embodiments of a method provided herein, the iDPCs on the scaffold are grafted onto a subject. In some embodiments, at least 100,000 iDPCs are grafted onto the subject. In some embodiments, at least 1,000,000 iDPCs are grafted onto the subject. In some embodiments, at least 10,000,000 iDPCs are grafted onto the subject. In some embodiments, iDPCs and dermal cells on the scaffold are grafted onto a subject. In some embodiments, at least 100,000 of a combination of iDPCs and dermal cells are grafted onto the subject. In some embodiments, at least 1,000,000 of a combination of iDPCs and dermal cells are grafted onto the subject. In some embodiments, at least 10,000,000 of a combination of iDPCs and dermal cells are grafted onto the subject.

Also provided herein is a method for treating a subject having a hair loss condition, comprising administering a composition comprising a population of non-DP cells; a population of iDPCs expressing one or more marker(s) selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2; and a DPCR system comprising one or more DPCR factor(s) selected from the group consisting of an EBF agent and a TWIST agent wherein the DPCR system causes transdifferentiation of one or more non-DP cell into one or more iDPC.

In some embodiments, compositions provided herein are administered to a subject in a method for treating a hair loss condition. For example, in some embodiments, the hair loss condition is selected from the group consisting of androgenic alopecia, alopecia areata, telogen effluvium, trichotillomania, traction alopecia, tinea capitis, cicatricial alopecia, burning or scarring. In some embodiments, the iDPCs are transdifferentiated from non-DP cells obtained from the subject. In some embodiments, the iDPCs are transdifferentiated from non-DP cells obtained from a subject different than the subject to which the composition is administered.

Also provided herein is a composition of iDPCs produced by the process comprising obtaining a population of iDPCs, obtaining a population of non-DP cells; contacting the population of non-DP cells with DPCR system comprising one or more DPCR factor(s) selected from the group consisting of an EBF agent and a TWIST agent; and incubating the population of non-DP cells in the presence of the DPCR system, wherein one or more non-DP cell is transdifferentiated into one or more iDPC.

### BRIEF DESCRIPTION OF THE DRAWINGS

FIG. 1 is a series of graphs showing qPCR analysis of canonical dermal papilla (DP) markers expressed by human iDPCs produced by overexpressing EBF1 and TWIST2 in cultured human epithelial cells (ECs). Expression of DP marker genes Alkaline Phosphatase (ALPL; FIG. 1A), EBF Transcription Factor 1 (EBF1; FIG. 1B), ETS Variant Transcription Factor 1 (ETV1; FIG. 1C), Fibroblast Growth Factor 7 (FGF7; FIG. 1D), Forkhead Box D1 (FOXD1; FIG. 1E), GLIS Family Zinc Finger 1 (GLIS1; FIG. 1F), Gremlin 2 (GREM2; FIG. 1G), Integrin Subunit Alpha 9 (ITGA9; FIG. 1H), Platelet Derived Growth Factor Receptor Alpha (PDGFRA; FIG. 1I), PR/SET domain 1 (PRDM1; FIG. 1J), Paired Related Homeobox 1 (PRRX1; FIG. 1K), R-spondin 4 (RSPO4; FIG. 1L), S100 Calcium Binding Protein B (S100B; FIG. 1M), SRY-box Transcription Factor 18 (SOX18; FIG. 1N), T-box Transcription Factor 18 (TBX18; FIG. 1O), Twist Family bHLH Transcription Factor 2 (TWIST2; FIG. 1P) were compared to negative control ECs. ND represents values that were Not Detected or detected above the CT cutoff of 30 cycles. *P<0.005, **P<0.0005 for one-tailed T-test.

FIG. 2 are micrographs showing whole-well images of alkaline phosphatase staining. Alkaline phosphatase activity was not detectable in negative control epithelial cells (EC) (FIG. 2A), while alkaline phosphatase activity was strongly detected in positive control DPs (FIG. 2B) and iDPCs overexpressing EBF1 and TWIST2 (FIG. 2C).

FIG. 3 are micrographs showing alkaline phosphatase staining (FIGS. 3A-3C) and graphs showing gene expression as assessed by qPCR (FIGS. 3D-3I) for iDPCs following overexpression of EBF1 and TWIST2 and negative control ECs. FIGS. 3A-3C show alkaline phosphatase staining in EC negative controls (FIG. 3A), iDPCs at passage 0 (P0, FIG. 3B), and iDPCs at passage 3 (P3, FIG. 3C). FIGS. 3D-3I show graphs of gene expression of negative control ECs and iDPCs at passage (P) 0, 1, 2, and 3 for ALPL (FIG. 3D), ITGA9 (FIG. 3E), PDGFRA (FIG. 3F), RSPO4 (FIG. 3G), SOX18 (FIG. 3H), and TBX18 (FIG. 3I) normalized to actin. ND represents values that were Not Detected or detected above the CT cutoff of 30 cycles.

FIGS. 4A-4E show flow cytometry plots of iDPCs overexpressing EBF1 and TWIST2 and FIGS. 4F-4J show flow cytometry negative control ECs. FIGS. 4A-4C and FIGS. 4F-4H show forward scatter (FSC) and side-scatter (SSC) gating parameters for cell size. FIG. 4D and FIG. 41 show propidium iodide (PI) staining for exclusion of dead cells. FIG. 4E and FIG. 4J show ITGA9 and PDGFR expression. The indicated rectangular gates and percentages indicate the ITGA9+/PDGFR+ cell population.

### DETAILED DESCRIPTION OF THE INVENTION

The present invention provides compositions and methods of using and producing a population of induced dermal papilla cells (iDPCs) from a population of non-dermal papilla (DP) cells. These methods and compositions find use in producing dermal papillae (DPs) and dermal sheath stem cells (DS) thereof for transplantation; as an experimental model for evaluating therapeutics; and as a source of lineage- and cell-specific products, and the like, for example, for use in treating human hair loss conditions such as androgenic alopecia, alopecia areata, telogen effluvium, trichotillomania, traction alopecia, tinea capitis, and cicatricial alopecia. Also provided are compositions and methods for screening candidate agents for activity in transdifferentiating non-DP cells into iDPCs. These and other objects, advantages, and features of the invention will become apparent to those persons skilled in the art upon reading the details of the subject compositions and methods as more fully described below.

Unless defined otherwise, all technical and scientific terms used herein have the meaning commonly understood by one of ordinary skill in the art to which this disclosure belongs. To facilitate an understanding of the present invention, a number of terms and phrases are defined below.

### Definitions

As used herein, the singular forms “a,” “an,” and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm).

A “stem cell” is a cell characterized by the ability of self-renewal through mitotic cell division and the potential to differentiate into the cell types that contribute to a tissue or an organ. Among mammalian stem cells, embryonic and somatic stem cells may be distinguished. The term “embryonic stem cell” is used to refer to the pluripotent stem cells of the inner cell mass of the embryonic blastocyst. These cells are capable of giving rise to an entire organism. The term “somatic stem cell” refers to any pluripotent or multipotent stem cell that differentiates into and maintains fetal, juvenile, and adult tissues. Unlike embryonic stem cells, somatic stem cells cannot give rise to an entire organism.

Pluripotent stem cells, which include embryonic stem cells, embryonic germ cells and induced pluripotent cells, can contribute to tissues of a prenatal, postnatal or adult organism.

The terms “primary cells,” “primary cell lines,” and “primary cultures,” are used interchangeably herein to refer to cells and cell cultures that have been derived from a subject and allowed to grow in vitro for a limited number of passages, i.e., splittings, of the culture.

The terms “treatment,” “treating,” “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient” are used interchangeably herein and refer to any mammalian subject for whom diagnosis, treatment, or therapy is desired, particularly humans.

### Dermal Papilla Cells and Dermal Sheath Stem Cells

As used herein, the term “dermal sheath stem cells” (DSC) refers to self-renewing, multipotent cells that give rise to dermal papillae at the base of the hair follicle. By self-renewing, it is meant that when DSC undergo mitosis, they are capable of producing at least one daughter cell that is a DSC. By multipotent it is meant that it is capable of giving rise to terminally differentiated dermal papillae that regulate hair cycling, growth, and maintenance of the hair follicle. DSCs are capable of contributing to a new hair structure in vivo when using techniques known to those skilled in the art. DSCs are not pluripotent, that is, they are not capable of giving rise to cells of other non-ectodermal organs.

As used herein, the terms “dermal papillae” (DP), “dermal papilla” (DP), and “dermal papilla cell” (DPC) refer to the downstream progeny of DSCs that arise during development. DPs have limited self-renewal capacity. By self-renewal, it is meant that when DPs undergo mitosis, they produce at least one daughter cell that is a DP. By limited self-renewal capacity, it is meant that DPs can self-renew for a limited number of mitoses (i.e., 1, 2, 3, 5, or 10 mitoses) before giving rise to two daughter cells that no longer undergo mitosis. DPs are unipotent, meaning that they are only capable of giving rise to the single DP cell type. DPs are not pluripotent, meaning they are not capable of giving rise to cells of other non-ectodermal lineages. DPs are capable of contributing to a new hair structure in vivo when using techniques known to those skilled in the art. Due to the fact that DPs have similar functional properties to DSCs, DSCs are commonly referred to as DPs and will be referred to here as such. The term “induced dermal papilla cells” (iDPC) encompasses DPs or DSCs that arise from non-DP cells by experimental manipulation. iDPCs exhibit the same phenotypic and functional properties as DPs or DSCs, as defined above.

The term “hair unit” encompasses a combination of epidermal cells and dermal cells. Epidermal cells may include epithelial cells, hair follicle stem cells, hair follicle progenitor cells, hair follicle cells, other cell types known to compose the epidermal cell layer, or cells that are functionally equivalent to those cell types. Dermal cells may comprise iDPCs, dermal papilla cells, dermal sheath cells, and other cell types known to compose the dermis. The hair unit may produce hair in a culture dish. The hair unit may also produce hair upon transplantation in vivo.

DPs express one or more marker(s) including ALPL, EBF1, ETV1, FGF7, FOXD1, GLIS1, GREM2, ITGA9, PDGFRA, PRDM1, PRRX1, RSPO4, S100B, SOX18, TBX18, and TWIST2, and other markers known to those skilled in the art. In some embodiments, DPs express one, two, three, four, five, six, seven, eight, nine, or all of the markers selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2. In some embodiments, expression levels of one or more of the markers selected from the group consisting of: ALPL, EBF1, ETV1, FGF7, FOXD1, GLIS1, GREM2, ITGA9, PDGFRA, PRDM1, PRRX1, RSPO4, S100B, SOX18, TBX18, and TWIST2 are modulated over time and/or depending on culture conditions. For example, DPs can express at least two-fold more, at least 3-fold more, at least 4-fold more, at least 5-fold more, at least 6-fold more, at least 7-fold more, at least 8-fold more, at least 9-fold more, or at least 10 fold more of one or more of the markers selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2 as compared to a population of non-DP cells. In an embodiment, expression of the one or more markers is measured using immunofluorescent staining (e.g., flow cytometry or immunohistochemistry), or western blot analysis. In a preferred embodiment, expression of the one or more markers is measured using flow cytometry. In some embodiments, expression levels of mRNA encoding the one or more markers is measured. For example, in some embodiments, mRNA levels are measured using, quantitative polymerase chain reaction (qPCR), reverse transcription polymerase chain reaction (RT PCR), RNA-Seq, or northern blot analysis. In a preferred embodiment, mRNA levels of the one or more markers is measured using qPCR. In some embodiments, DPs maintain expression of one or more of the markers selected from the group consisting of ALPL, EBF1, ETV1, FGF7, FOXD1, GLIS1, GREM2, ITGA9, PDGFRA, PRDM1, PRRX1, RSPO4, S100B, SOX18, TBX18, and TWIST2, over at least one cell passage, for example, at least one cell passage, at least two cell passages, at least 3 cell passages, at least 4 cell passages, or at least 5 cell passages.

### Non-Dermal Papilla Cells

As used herein, the term “non-dermal papilla (DP) cell” encompasses any cell in an organism that cannot give rise to cells composing the dermal sheath or dermal cup under normal physiological conditions. This term may encompass other stem cells, hematopoietic cells, epidermal cells, epithelial cells, dermal cells, or any other cell type that cannot give rise to hair follicle cells under normal physiological conditions absent genetic manipulation or wounding (for example, cutting, burning or scarring). The non-DP cells may be from any mammal, including humans, primates, domestic and farm animals, and zoo, laboratory or pet animals, such as dogs, cats, cattle, horses, sheep, pigs, goats, rabbits, rats, mice etc. In some embodiments, non-DP cells are human or murine cells and are dermal cells (e.g. fibroblasts, smooth muscle cells, or connective tissue cells), epidermal cells, epithelial cells, adipocytes, or hematopoietic cells.

In some embodiments, non-DP cells may be established cell lines or they may be primary cells, where “primary cells,” “primary cell lines,” and “primary cultures” are used interchangeably herein to refer to cells and cells cultures that have been derived from a subject and allowed to grow in vitro for a limited number of passages. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro. The non-DP cells may be isolated from fresh or frozen cells, which may be from a neonate, a juvenile or an adult, and from tissues including skin, muscle, bone marrow, peripheral blood, umbilical cord blood, spleen, bladder, liver, pancreas, lung, intestine, stomach, adipose, and other differentiated tissues. The tissue may be obtained by biopsy or apheresis from a live donor or obtained from a dead or dying donor within about 48 hours of death, or freshly frozen tissue, tissue frozen within about 12 hours of death and maintained at below about −20° C., usually at about liquid nitrogen temperature (−190° C.) indefinitely. For isolation of cells from tissue, an appropriate solution may be used for dispersion or suspension. Such a solution will generally be a balanced salt solution, e.g., normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

### Transdifferentiation and Dermal Papilla Cell Reprogramming System

The term “transdifferentiation” refers to the deliberate induction of a transition of a cell or group of cells to a different cell type using genetic or biochemical manipulations such as introduction of an exogenously derived gene or protein into the cell. This is distinct from the normal processes of differentiation or de-differentiation, which may be artificially induced through standard cell culture procedures or treatment with growth factors or other signaling molecules.

The terms “dermal papilla cell reprogramming factors” or “DPCR factors” refer to one or more, i.e., a cocktail, of biologically active factors that act on a non-DP cell to promote reprogramming, i.e., transdifferentiation, of the targeted cell into a iDPC. As used herein, the term “DPCR system” refers to reagents and culture conditions that promote the reprogramming, i.e., transdifferentiation, of non-DP cells to iDPCs where the non-DP cells may be somatic cells or may be pluripotent cells. A DPCR system comprises one or more, i.e., a cocktail, of non-DP cell-to-DPCR factors. A DPCR system may also optionally comprise other reagents, such as agents that promote cell reprogramming, agents that promote the survival and differentiation of DPs, agents that promote the differentiation of subtypes of DPs, agents that promote the survival and differentiation of DSCs, agents that promote the differentiation of subtypes of DSCs, and the like, as known in the art. A DPCR system does not induce a non-DP cell to become pluripotent, e.g., an induced pluripotent stem cell (iPSC), in the course of conversion into iDPCs. In other words, a DPCR system induces the transdifferentiation of non-DP cells of one lineage into iDPCs, or induces pluripotent cells to become iDPCs.

In some embodiments, DPCR systems comprise conditions that induce the conversion of non-DP cells into iDPCs from a subject; or induce the conversion of a pluripotent cell into an iDPC. In some embodiments, the non-DP cells are contacted in vitro with the DPCR system comprising of one or more non-DP cell-to-DP reprogramming factors (DPCR factors). In some embodiments, the one or more DPCR factors are provided as nuclear acting polypeptides. In other words, the subject cells are contacted with DPCR polypeptides that act in the nucleus.

To promote transport of DPCR polypeptides across the cell membrane, DPCR polypeptide sequences may be fused to a polypeptide permeant domain. A number of permeant domains are known in the art and may be used in the nuclear acting polypeptides of the present invention, including peptides, peptidomimetics, and non-peptide carriers. For example, a permeant peptide may be derived from the third alpha helix of Drosophila melanogaster transcription factor Antennapedia, referred to as penetratin, which comprises the amino acid sequence RQIKIWFQNRRMKWKK (SEQ ID NO: 1). As another example, the permeant peptide comprises the HIV-1 tat basic region amino acid sequence, which may include, for example, amino acids 49-57 of the naturally-occurring tat protein (SEQ ID NO:2). Other permeant domains include poly-arginine motifs, for example, the region of amino acids 34-56 of HIV-1 rev protein (SEQ ID NO:3), nona-arginine (SEQ ID NO:4), octa-arginine (SEQ ID NO:5), and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9; SEQ ID NO:4) sequence is one of the more efficient PTDs that have been characterized (Wender et al. 2000; Uemura et al. 2002).

The DPCR polypeptides may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Other methods of preparing polypeptides in a cell-free system include, for example, those methods taught in U.S. Application Ser. No. 61/271,000.

The DPCR polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein. DPCR polypeptides may be produced recombinantly not only directly, but also as a fusion polypeptide with a heterologous polypeptide, e.g., a polypeptide having a specific cleavage site at the N-terminus of the mature protein or polypeptide. Expression vectors usually contain a selection gene, also termed a selectable marker. This gene encodes a protein necessary for the survival or growth of transformed host cells grown in a selective culture medium.

Following purification by commonly known methods in the art, DPCR polypeptides are provided to the subject cells by standard protein transduction methods. In some cases, the protein transduction method includes contacting cells with a composition containing a carrier agent and at least one purified DPCR polypeptide. Examples of suitable carrier agents and methods for their use include, but are not limited to, commercially available reagents such as Chariot™ (Active Motif, Inc., Carlsbad, Calif) described in U.S. Pat. No. 6,841,535; Bioport™ (Gene Therapy Systems, Inc., San Diego, Calif.), GenomeONE (Cosmo Bio Co., Ltd., Tokyo, Japan), and ProteoJuice™ (Novagen, Madison, Wis.), or nanoparticle protein transduction reagents.

In other embodiments, the one or more DPCR factors are nucleic acids (e.g., polynucleotides) encoding DPCR polypeptides, i.e., DPCR nucleic acids. Nucleic acids can be deoxyribonucleic acids (DNA), ribonucleic acids (RNA) or functionally similar derivatives. Vectors used for providing DPCR nucleic acids to the subject cells will typically comprise suitable promoters for driving the expression, that is, transcriptional activation, of the nucleic acids. This may include ubiquitously acting promoters, for example, the CMV-β-actin promoter, or inducible promoters, such as promoters that are active in particular cell populations or that respond to the presence of drugs such as tetracycline. By transcriptional activation, it is intended that transcription will be increased above basal levels in the target cell by at least about 10-fold, by at least about 100-fold, more usually by at least about 1000-fold. In addition, vectors used for providing the nucleic acids may include genes that must later be removed, e.g., using a recombinase system such as Cre/Lox, or genes that cause the cells that express them to be destroyed, e.g., by including genes that allow selective toxicity such as herpesvirus TK, bcl-xs, etc.

DPCR nucleic acids may be provided directly to the subject cells. In other words, the cells are contacted with vectors comprising DPCR nucleic acids such that the vectors are taken up by the cells. Methods for contacting cells with nucleic acid vectors, such as electroporation, calcium chloride transfection, and lipofection, are well known in the art. Vectors that deliver nucleic acids in this manner are usually maintained episomally, e.g., as plasmids or minicircle DNAs.

Alternatively, the nucleic acid may be provided to the subject cells via a virus. In other words, the cells are contacted with viral particles comprising the DPCR nucleic acids. Retroviruses, for example, lentiviruses, are particularly suitable for such methods. Commonly used retroviral vectors are “defective,” i.e., unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g., MMLV, are capable of infecting most murine and rat cell types, and are generated by using ecotropic packaging cell lines such as BOSC23 (Pear et al. (1993) Proc. Natl. Acad. Sci. 90:8392-8396). Retroviruses bearing amphotropic envelope protein, e.g., 4070A (Danos et al., supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) Proc. Natl. Acad. Sci. 85:6460-6464). Retroviruses packaged with xenotropic envelope protein, e.g., AKR env, are capable of infecting most mammalian cell types, except murine cells. The appropriate packaging cell line may be used to ensure that the subject cells are targeted by the packaged viral particles. Methods of introducing the retroviral vectors comprising DPCR nucleic acids into packaging cell lines and of collecting the viral particles that are generated by the packaging lines are well known in the art.

The effective amount of a DPCR system that may be used to contact the non-DP cells is an amount that induces at least 0.01% of the cells of the culture to increase expression of one or more genes known in the art to become more highly expressed upon the acquisition of a DP fate, e.g., ALPL, EBF1, ETV1, FGF7, FOXD1, GLIS1, GREM2, ITGA9, PDGFRA, PRDM1, PRRX1, RSPO4, S100B, SOX18, TBX18, and TWIST2.

An effective amount is the amount that induces an increase in expression of these genes that is, e.g., about 1.5 fold, 2 fold, 3 fold, 4 fold, 6 fold, or 10 fold greater than the level of expression observed in the absence of the DPCR system. The level of gene expression can be readily determined by any of a number of well-known methods in the art, e.g., by measuring RNA levels by methods such as, but not limited to, RT-PCR, quantitative RT-PCR, RNA-Seq, and Northern blot; and by measuring protein levels by methods such as, but not limited to, Western blot, ELISA, and fluorescence activated cell sorting.

It is noted here that the contacted non-DP cells do not need to be cultured under methods known in the art to promote pluripotency in order to be converted into iDPCs. By pluripotency, it is meant that the cells have the ability to differentiate into all types of cells in an organism.

For the DPCR system, the efficiency of reprogramming may be determined by assaying the number of iDPCs that develop in the cell culture, e.g. by assaying the number of cells that express genes that are expressed by DPs, e.g., ALPL, EBF1, ETV1, FGF7, FOXD1, GLIS1, GREM2, ITGA9, PDGFRA, PRDM1, PRRX1, RSPO4, S100B, SOX18, TBX18, and TWIST2.

For the DPCR system, following the methods of the invention, the contacted non-DP cells will be converted into iDPCs or DSCs at an efficiency of reprogramming/efficiency of conversion that is at least about 0.01% of the total number of non-DP cells cultured initially, e.g., about 0.01%, 0.05%, 0.1%, 0.5%, 1%, 2%, 3%, 4%, 5%, 6%, 7%, 8%, 9%, 10%, 12%, 14%, 16%, 20% or more.

At times, depending on the age of the donor, the origin of the tissue, or the culture conditions, higher efficiencies may be achieved. This efficiency of reprogramming is an enhanced efficiency over that which may be observed in the absence of DPCR system(s). By enhanced, it is meant that the non-DP cells cultures have the ability to give rise to the desired cell type that is at least 150% greater than the ability of a non-DP cell culture that was not contacted with the DPCR factor(s), e.g., at least 150%, at least 200%, at least 300%, at least 400%, at least 600%, at least 800%, at least 1000%, or at least 2000% of the ability of the uncontacted population. In other words, for cells treated with DPCR system, the culture of non-DP cells produces at least 1.5 fold, at least 2-fold, at least 3-fold, at least 4-fold, at least 6-fold, at least 10-fold, at least 20-fold, at least 30-fold, at least 50-fold, at least 100-fold, or at least 200-fold the number of iDPCs that are produced by a population of non-DP cells that are not contacted with the DPCR system.

In some cases, genes may be introduced into the non-DP cells or the cells derived therefrom, i.e., iDPCs or differentiated progeny cells, prior to transferring to a subject for a variety of purposes, for example, but not limited to, replacing genes having a loss of function mutation (e.g., loss of function mutations in ALPL, EBF1, ETV1, FGF7, FOXD1, GLIS1, GREM2, ITGA9, PDGFRA, PRDM1, PRRX1, RSPO4, S100B, SOX18, TBX18, or TWIST2), or providing marker genes (e.g., Green Fluorescent Protein, or antibiotic resistance genes). Alternatively, vectors are introduced that express antisense mRNA or ribozymes, thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as Bcl-2. Various techniques known in the art may be used to introduce nucleic acids into the target cells, e.g., electroporation, calcium precipitated DNA, fusion, transfection, lipofection, infection and the like, as discussed above. The particular manner in which the nucleic acids are introduced is not critical to the practice of the invention.

To confirm that non-DP cells or the cells derived therefrom, have been genetically modified, i.e., transdifferentiated to iDPCs or differentiated progeny cells, various techniques may be employed. The genome, transcriptome, or proteome of the cells may be restricted and used with or without amplification. Polymerase chain reaction; gel electrophoresis; restriction analysis; Southern, Northern, and Western blots; sequencing; or the like, may all be employed to confirm genetic manipulation. Various tests in vitro and in vivo may be employed to ensure that the iDPC or differentiated progeny cell phenotype of the derived cells has been maintained.

### Dermal Papilla Cell Reprogramming (DPCR) Factors

The following describes the DPCR factors for the DPCR system described in the previous section.

DPCR factors are biologically active factors that act on a cell to alter transcription so as to convert the cell into a DP, i.e., an iDPC. DPCR factors are provided to somatic or pluripotent cells in the context of a DPCR system. Examples of DPCR factors include an EBF agent and a TWIST agent.

The term EBF agent is used to refer to EBF (also called Early B-Cell Factor) polypeptides, functional fragments thereof, and the nucleic acids that encode them. The term EBF agent may also refer to polypeptides of EBF-related proteins or proteins that regulate EBF activity and the nucleic acids that encode them. In some embodiments, an EBF polypeptide or functional fragment thereof is fused to a permeant domain. EBF agents may also refer to small molecules that modulate EBF expression and/or activity.

The terms “EBF gene product,” “EBF polypeptide,” and “EBF protein” are used interchangeably herein to refer to native sequence EBF polypeptides, EBF polypeptide variants, EBF polypeptide fragments and chimeric EBF polypeptides that can modulate transcription. Native sequence EBF polypeptides include the proteins EBF1 (GenBank Accession Nos. NM_001290360.2 and NP_001277289.1); EBF2 (GenBank Accession Nos. NM_022659.3 and NP_073150.2); EBF3 (GenBank Accession Nos. NM_001005463.2 and NP_001005463.1); and EBF4 (GenBank Accession Nos. NM_001110514.1 and NP_001103984.1). EBF polypeptides, e.g., those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as reprogramming factors in the present invention, as do nucleic acids encoding these polypeptides or their transcriptionally active domains and vectors comprising these nucleic acids. In certain embodiments, the EBF agent is an EBF1 agent.

The term TWIST agent is used to refer to TWIST polypeptides, functional fragments thereof, and the nucleic acids that encode them. The term TWIST agent may also refer to polypeptides of TWIST-related proteins or proteins that regulate TWIST activity and the nucleic acids that encode them. In some embodiments, a TWIST polypeptide or functional fragment thereof is fused to a permeant domain. TWIST agents may also refer to small molecules that modulate TWIST expression and/or activity. TWIST polypeptides are members of the TWIST bHLH transcription factor family.

The terms “TWIST gene product,” “TWIST polypeptide,” and “TWIST protein” are used interchangeably herein to refer to native sequence TWIST polypeptides, TWIST polypeptide variants, TWIST polypeptide fragments and chimeric TWIST polypeptides that can modulate transcription. Native sequence TWIST polypeptides include the proteins TWIST1 (GenBank Accession Nos. NM_000474.3 and NP_000465.1); and TWIST2 (GenBank Accession Nos. NM_001271893.3 and NP_001258822.1). TWIST polypeptides, e.g., those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as reprogramming factors in the present invention, as do nucleic acids encoding these polypeptides or their transcriptionally active domains and vectors comprising these nucleic acids. In certain embodiments, the TWIST agent is a TWIST2 agent.

In some embodiments, only one DPCR factor is provided, e.g., an EBF agent or a TWIST agent.

In some embodiments, a set of at least two agents is provided, e.g., an EBF agent and a TWIST agent.

All GenBank Accession No. sequences disclosed herein existed in the National Institutes of Health (NIH) genetic sequence database (GenBank) as at the date of filing of this priority application.

### Cell Culture

Cells contacted in vitro with the DPCR system of reagents may be incubated in the presence of the reagent(s) for about 1 hour to about 8 weeks, e.g., 1 hour to 8 weeks, 2 hours to 8 weeks, 4 hours to 8 weeks, 6 hours to 8 weeks, 12 hours to 8 weeks, 18 hours to 8 weeks, 24 hours to 8 weeks, 48 hours to 8 weeks, 72 hours to 8 weeks, 96 hours to 8 weeks, 1 week to 8 weeks, 2 weeks to 8 weeks, 4 weeks to 8 weeks, 1 hour to 6 weeks, 2 hours to 6 weeks, 4 hours to 6 weeks, 6 hours to 6 weeks, 12 hours to 6 weeks, 18 hours to 6 weeks, 24 hours to 6 weeks, 48 hours to 6 weeks, 72 hours to 6 weeks, 96 hours to 6 weeks, 1 week to 6 weeks, 2 weeks to 6 weeks, 4 weeks to 6 weeks, 1 hour to 4 weeks, 2 hours to 4 weeks, 4 hours to 4 weeks, 6 hours to 4 weeks, 12 hours to 4 weeks, 18 hours to 4 weeks, 24 hours to 4 weeks, 48 hours to 4 weeks, 72 hours to 4 weeks, 96 hours to 4 weeks, 1 week to 4 weeks, 2 weeks to 4 weeks, 1 hour to 2 weeks, 2 hours to 2 weeks, 4 hours to 2 weeks, 6 hours to 2 weeks, 12 hours to 2 weeks, 18 hours to 2 weeks, 24 hours to 2 weeks, 48 hours to 2 weeks, 72 hours to 2 weeks, 96 hours to 2 weeks, or 1 week to 2 weeks. For example, in some embodiments, a population of non-DP cells is incubated in the presence of the DPCR system for a period of 1 hour to 6 weeks. In some embodiments, replacement of the DPCR system may be replaced with a frequency of about every day to about every 14 days, e.g., every day, 1.5 days, 2 days, 3 days, 4 days, 5 days, 6 days, 7 days, or 14 days. The reagent(s) may be provided to the subject cells one or more times, e.g., one time, twice, three times, or more than three times, and the cells allowed to incubate with the reagent(s) for some amount of time following each contacting event, e.g., 24 hours to 6 weeks, after which time the media is replaced with fresh media and the cells are cultured further.

After contacting the non-DP cells with the DPCR system, the contacted cells may be cultured so as to promote the survival of DPs, DSCs, or hair follicles. Methods and reagents for culturing cells, DPs, DSCs, and differentiated hair follicles and for isolating DPs, DSCs, and differentiated hair follicles are well known in the art, any of which may be used in the present invention to grow and isolate the iDPCs and/or differentiated progeny.

For example, the non-DP cells (either pre- or post-contacting with the DPCR systems) may be plated on Matrigel or a similar basement membrane extract, a synthetic hydrogel, gelatin, collagen, laminin or other substrate as known in the art. The cells may be cultured in media such as DMEM, supplemented with factors. Alternatively, the contacted cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or stromal cells associated with DP survival and differentiation.

In some embodiments a population of iDPCs are dissociated to form a cell suspension. Cell dissociation methods are well known in the art and can include enzymatic dissociation (e.g. trypsin dissociation), chemical dissociation (e.g., EDTA or EGTA dissociation), or mechanical dissociation. In some embodiments, a population of dermal cells is added to the dissociated population of iDPCs. For example, in some embodiments, the population of dermal cells are fibroblasts, smooth muscle cells, connective tissue cells, dermal papilla cells, and adipocytes. In some embodiments, a population of epidermal cells is added to the dissociated population of iDPCs. For example, in some embodiments, the population of epidermal cells comprise epithelial cells, hair follicle stem cells, and/or hair follicle progenitor cells.

In some embodiments, iDPCs are cultured on a scaffold. For example, a scaffold can include, but is not limited to, a plastic, a Matrigel or similar basement membrane extract, gelatin, collagen, or laminin matrix. In some embodiments, iDPCs are cultured on the scaffold for 30 minutes to 48 hours, e.g., 30 minutes to 24 hours, 30 minutes to 12 hours, 30 minutes to 6 hours, 30 minutes to 3 hours, 30 minutes to 2 hours, 30 minutes to 1 hour, 1 hour to 48 hours, 1 hour to 24 hours, 1 hour to 12 hours, 1 hour to 6 hours, 1 hour to 3 hours, or 1 hour to 2 hours. In some embodiments, iDPCs are cultured in combination with dermal cells on the scaffold for 30 minutes to 48 hours, e.g., 30 minutes to 24 hours, 30 minutes to 12 hours, 30 minutes to 6 hours, 30 minutes to 3 hours, 30 minutes to 2 hours, 30 minutes to 1 hour, 1 hour to 48 hours, 1 hour to 24 hours, 1 hour to 12 hours, 1 hour to 6 hours, 1 hour to 3 hours, or 1 hour to 2 hours. In some embodiments, iDPCs are cultured in combination with epidermal cells on the scaffold for 30 minutes to 48 hours, e.g., 30 minutes to 24 hours, 30 minutes to 12 hours, 30 minutes to 6 hours, 30 minutes to 3 hours, 30 minutes to 2 hours, 30 minutes to 1 hour, 1 hour to 48 hours, 1 hour to 24 hours, 1 hour to 12 hours, 1 hour to 6 hours, 1 hour to 3 hours, or 1 hour to 2 hours. In some embodiments, iDPCs are cultured in combination with epidermal and dermal cells on the scaffold for 30 minutes to 48 hours, e.g., 30 minutes to 24 hours, 30 minutes to 12 hours, 30 minutes to 6 hours, 30 minutes to 3 hours, 30 minutes to 2 hours, 30 minutes to 1 hour, 1 hour to 48 hours, 1 hour to 24 hours, 1 hour to 12 hours, 1 hour to 6 hours, 1 hour to 3 hours, or 1 hour to 2 hours.

### Methods of Treatment

iDPCs or differentiated progeny cells produced by the above in vitro methods may be used in cell replacement or cell transplantation therapy to treat diseases. Specifically, iDPCs and/or differentiated progeny may be transferred to subjects suffering from a wide range of diseases, conditions, or disorders with a hair loss component, i.e., with hair loss symptoms, for example to reconstitute or supplement hair forming units in a recipient. The therapy may be directed at treating the cause of the disease; or alternatively, the therapy may be to treat the effects of the disease or condition. For example, the therapy may be directed at replacing hair follicles whose death or senescence caused the disease.

The iDPCs and/or differentiated progeny cells may be transferred to, or close to, an injured site in a subject; or the cells can be introduced to the subject in a manner allowing the cells to migrate, or home, to the injured site. The transferred cells may advantageously replace the damaged or injured cells and allow improvement in the overall condition of the subject. In some instances, the transferred cells may stimulate tissue regeneration or repair.

In some cases, the iDPCs and/or differentiated progeny cells or a sub-population of iDPCs and/or differentiated progeny cells may be purified or isolated from the rest of the cell culture prior to transferring to the subject. In other words, one or more steps may be executed to enrich for the iDPCs and/or differentiated progeny cells or a subpopulation of iDPCs or differentiated progeny cells, i.e., to provide an enriched population of iDPCs or differentiated progeny cells or subpopulation of iDPCs or differentiated progeny cells. In some cases, one or more antibodies specific for a marker of cells of the DPs or differentiated progeny lineages or a marker of a sub-population of cells of DPs or differentiated progeny lineages are incubated with the cell population and those bound cells are isolated. In other cases, the iDPCs or differentiated progeny cells or a sub-population of the iDPCs or differentiated progeny cells express a marker that is a reporter gene, e.g., EGFP, dsRED, lacz, and the like, that is under the control of a DP-specific, DSC-specific, or differentiated hair cell-specific promoter which is then used to purify or isolate the iDPCs or differentiated progeny cells or a subpopulation thereof.

By a marker it is meant that, in cultures comprising non-DP cells that have been reprogrammed to become iDPCs, the marker is expressed by the cells of the culture that will develop, are developing, and/or have developed into dermal papilla cells. It will be understood by those of skill in the art that the stated expression levels reflect detectable amounts of the marker protein on or in the cell. A cell that is negative for staining (the level of binding of a marker-specific reagent is not detectably different from an isotype matched control) may still express minor amounts of the marker. And while it is commonplace in the art to refer to cells as “positive” or “negative” for a particular marker, actual expression levels are a quantitative trait. The number of molecules on the cell surface can vary by several logs, yet still be characterized as “positive”.

Cells of interest, i.e., cells expressing the marker of choice, may be enriched for, that is, separated from the rest of the cell population, by a number of methods that are well known in the art. For example, flow cytometry, e.g., fluorescence activated cell sorting (FACS), may be used to separate the cell population based on the intrinsic fluorescence of the marker, or the binding of the marker to a specific fluorescent reagent, e.g., a fluorophore-conjugated antibody, as well as other parameters such as cell size and light scatter. In other words, selection of the cells may be affected by flow cytometry. Although the absolute level of staining may differ with a particular fluorochrome and reagent preparation, the data can be normalized to a control. To normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining above the brightness of an isotype matched control, but are not as intense as the most brightly staining cells normally found in the population. An alternative control may utilize a substrate having a defined density of marker on its surface, for example a fabricated bead or cell line, which provides the positive control for intensity.

Other methods of separation, i.e., methods by which selection of cells may be affected, based upon markers include, for example, magnetic activated cell sorting (MACS), immunopanning, and laser capture microdissection.

Enrichment of iDPC populations or a subpopulation of iDPCs or differentiated progeny cells may be performed about 3 days or more after contacting the non-DP cells with the DPCR system, e.g., 3 days, 4 days, 5 days, 6 days, 7 days, 10 days, 14 days, 21 days, 30 days or 40 days after contacting the non-DP cells with the DPCR system. Populations that are enriched by selecting for the expression of one or more markers will usually have at least about 80% cells of the selected phenotype, more usually at least 90% cells and may be 95% of the cells, or more, of the selected phenotype. Subjects in need of hair transplantation therapy, e.g., a subject suffering from a condition associated with the loss of hair or with aberrantly functioning hair follicles, could especially benefit from therapies that utilize cells derived by the methods of the invention. Examples of such diseases, disorders and conditions include disorders which affect hair growth including androgenic alopecia, alopecia areata, telogen effluvium, trichotillomania, traction alopecia, tinea capitis, cicatricial alopecia. Examples of other conditions which may affect hair growth include scarring and trauma to the skin including chemical or heat induced burns. In some approaches, the reprogrammed non-DP cells, i.e., iDPCs or differentiated progeny may be transplanted directly to an injured site to treat a hair loss condition or in combination with such techniques known in the art such as follicular unit transplantation, follicular unit strip transplantation, and the like. In other approaches, the cells derived by the methods of the invention are engineered to respond to cues that can target their migration into existing epidermis, hair follicles, dermis or components of the skin. The iDPCs may be administered in any physiologically acceptable medium. They may be provided prior to differentiation, i.e., they may be provided in an undifferentiated state and allowed to differentiate in vivo, or they may be allowed to differentiate for a period of time ex vivo and provided following differentiation. They may be cultured and/or differentiated prior to administration and they can be cultured and/or differentiated together as a co-culture prior to administration e.g., to support their growth and/or organization in the tissue to which they are being transplanted. They may be provided alone or with a suitable substrate, matrix (e.g., plastic, Matrigel or similar basement membrane extract, gelatin, collagen, or laminin matrix), in combination, or in combination with cells such as dermal or epidermal cells e.g., to support their growth and/or organization in the tissue to which they are being transplanted. For example, in some embodiments dermal cells are fibroblasts, smooth muscle cells, connective tissue cells, dermal papilla cells, or adipocytes. In some embodiments, at least 1×10⁵ cells, at least 1×10⁶ cells, or at least 1×10⁷ cells are administered to a subject. The cells may be introduced to the subjects by topical surgery, injection, or the like. Examples of methods for local delivery include follicular unit transplantation, follicular unit strip therapy, or by implanting a device upon which the cells have been reversibly affixed. For example, in some embodiments, a scaffold supporting at least 100,000 iDPCs, at least 1×10⁶ iDPCs, or at least 1×10⁷ iDPCs is grafted onto the subject. In some embodiments, a scaffold supporting at least 100,000 iDPCs and epidermal cells, at least 1×10⁶ iDPCs and epidermal cells, or at least 1×10⁷ iDPCs and epidermal cells is grafted onto the subject. In some embodiments, a scaffold supporting at least 100,000 iDPCs, dermal, and epidermal cells, at least 1×10⁶ iDPCs, dermal, and epidermal cells, or at least 1×10⁷ iDPCs, dermal, and epidermal cells is grafted onto the subject.

The number of administrations of treatment to a subject may vary. Introducing the iDPCs and/or differentiated progeny cells into the subject may be a one-time event; but in certain situations, such treatment may elicit improvement for a limited period of time and require an on-going series of repeated treatments. In other situations, multiple administrations of the iDPCs and/or differentiated progeny cells may be required before an effect is observed. The exact protocols depend upon the disease or condition, the stage of the disease and parameters of the individual subject being treated.

### Experimental or Screening Uses

Additionally or alternatively, iDPCs and/or differentiated progeny produced by the above in vitro methods may be used as a basic research or drug discovery tool, for example to evaluate the phenotype of a genetic disease, e.g., to better understand the etiology of the disease, to identify target proteins for therapeutic treatment, to identify candidate agents with disease-modifying activity, i.e., an activity in modulating the survival or function of DPs or differentiated progeny cells in a subject suffering from a hair loss disease or disorder, e.g., to identify an agent that will be efficacious in treating the subject. For example, a candidate agent may be added to a cell culture comprising iDPC and/or differentiated progeny cells derived from the subject's somatic cells, and the effect of the candidate agent assessed by monitoring output parameters such as iDPCs or differentiated progeny cell survival, the ability of the iDPCs or differentiated progeny cells to form hair or promote hair growth, the extent to which the iDPCs and/or differentiated progeny cells continuously produce hair, and the like, by methods described herein and in the art.

Parameters are quantifiable components of cells, particularly components that can be accurately measured, desirably in a high throughput system. A parameter can be any cell component or cell product including a cell surface determinant, receptor, protein or conformational or posttranslational modification thereof, lipid, carbohydrate, organic or inorganic molecule, nucleic acid, e.g., mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to one or a plurality of cell samples, usually in conjunction with cells lacking the agent. The change in parameters in response to the agent is measured, and the result evaluated by comparison to reference cultures, e.g., in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e., at zero concentration or below the level of detection of the agent or at or below the concentration of agent that does not give a detectable change in the phenotype.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Generation and Validation of Human Induced Dermal Papilla Cells

Induced dermal papilla cells (iDPCs) were produced by transducing the nucleotides encoding EBF1 and TWIST2 in lentiviral vectors containing tetracycline-inducible promoters in dermal papilla cell (DPC) medium. ECs were incubated with these viral particles for 12-24 hours to facilitate transduction. After this period, medium was replaced with fresh DPC medium supplemented with tetracycline. Cells were cultured further for another 1-4 weeks in DPC medium supplemented with tetracycline with the culture medium being replaced every 1-3 days. During this 1-4 week period, greater than 0.1% of transdifferentiating cells acquired a dermal cell like morphology and were termed putative iDPCs. Cultures in DPC medium were passaged as a mixed population of cells upon confluence. iDPCs continued to grow in DPC medium and were collected or frozen in cell freezing medium upon confluence.

Quantitative polymerase chain reaction (qPCR) analysis was used to measure expression of known DP markers (ALPL, EBF1, ETV1, FGF7, FOXD1, GLIS1, GREM2, ITGA9, PDGFRA, PRDM1, PRRX1, RSPO4, S100B, SOX18, TBX18, and TWIST2) normalized to the house-keeping gene actin. To conduct qPCR analysis, mRNA was isolated from iDPCs and negative control epithelial cells with Qiagen RNeasy kits according to manufacturer instructions. cDNA was produced with BioRad iScript Reverse Transcription Mix according to manufacturer instructions. qPCR was conducted on a BioRad C1000™ instrument with BioRad SsoAdvanced Universal SYBR™ Green SuperMix according manufacturer instructions and with the following amplification protocol: 95C for 30 seconds followed by 40 cycles of [95° C. for 15 seconds, 60° C. for 30 seconds]. Primer nucleotide sequences used for qPCR assays are listed in Table 1 below.

TABLE 1
PRIMERS FOR qPCR ANALYSIS OF DP MARKER GENES

| Gene | Sense primer | Antisense primer |
|---|---|---|
| Actin | ccaaccgcgagaagatga | ccagaggcgtacagggatag |
| | (SEQ ID NO: 6) | (SEQ ID NO: 7) |
| ALPL | gacggacccgtcactctc | gtgcccgtggtcaattct |
| | (SEQ ID NO: 8) | (SEQ ID NO: 9) |
| EBF1 | caccgaccttgcaaatactga | tgggaggtacaactttaacca |
| | (SEQ ID NO: 10) | (SEQ ID NO: 11) |
| ETV1 | gcatttaaaccttttactgctgtg | tcagcatgaaagagaatattacagaaa |
| | (SEQ ID NO: 12) | (SEQ ID NO: 13) |
| FGF7 | aagggacccaagagatgaaga | cctttgattgccacaattcc |
| | (SEQ ID NO: 14) | (SEQ ID NO: 15) |
| FOXD1 | gcaaagccgacctatgctac | gagcctctagtgcctggaca |
| | (SEQ ID NO: 16) | (SEQ ID NO: 17) |
| GLIS1 | cctccgtgcacctagcag | cagctcaagctcggatgg |
| | (SEQ ID NO: 18) | (SEQ ID NO: 19) |
| GREM2 | cagagggagagggagagagag | tccagaacatcctgcaatga |
| | (SEQ ID NO: 20) | (SEQ ID NO: 21) |
| ITGA9 | cagttcatgtcccgcgccaa | gcctcgaagaccaccgtcac |
| | (SEQ ID NO: 22) | (SEQ ID NO: 23) |
| PDGFRA | cacacagggttgtacacttgct | aaggctacatctgggtctgg |
| | (SEQ ID NO: 24) | (SEQ ID NO: 25) |
| PRDM1 | gcctgtaggaagtggcttgt | gaggtgaggggaaaccattt |
| | (SEQ ID NO: 26) | (SEQ ID NO: 27) |
| PRRX1 | tgaaattttgattcagacatccat | tttgggtttatgggttcagg |
| | (SEQ ID NO: 28) | (SEQ ID NO: 29) |
| RSPO4 | tttggcccaccagaacac | ggtccttcctgcccttct |
| | (SEQ ID NO: 30) | (SEQ ID NO: 31) |
| S100B | caacaatgagctttcccattt | cattgtccagtgtttccatga |
| | (SEQ ID NO: 32) | (SEQ ID NO: 33) |
| SOX18 | gtatgtttggttccatgtcacag | ggacacgtgggaactcca |
| | (SEQ ID NO: 34) | (SEQ ID NO: 35) |
| TBX18 | tctgcagtgtcccatgaagt | cagcatcagggtaggtagca |
| | (SEQ ID NO: 36) | (SEQ ID NO: 37) |
| TWIST2 | catgtccgcctcccacta | cgaatgcatcccaattcc |
| | (SEQ ID NO: 38) | (SEQ ID NO: 39) |

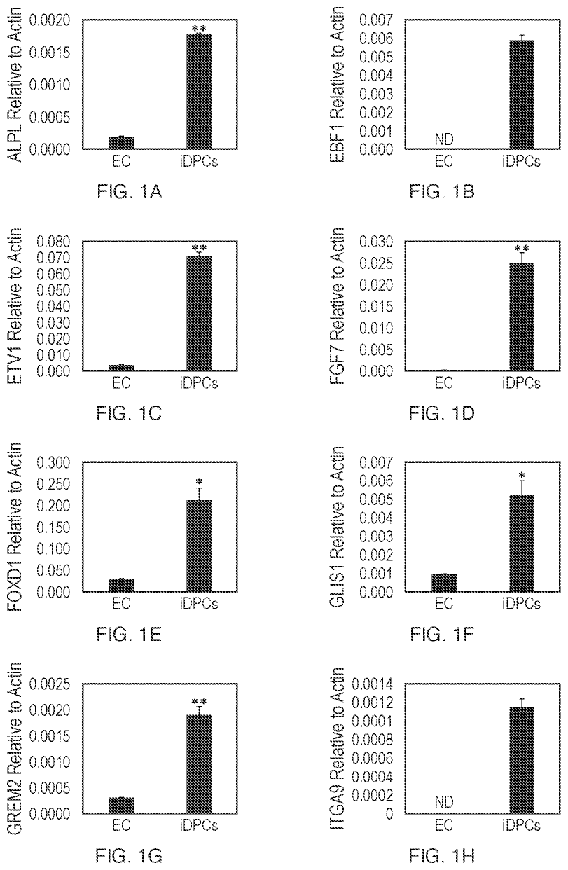

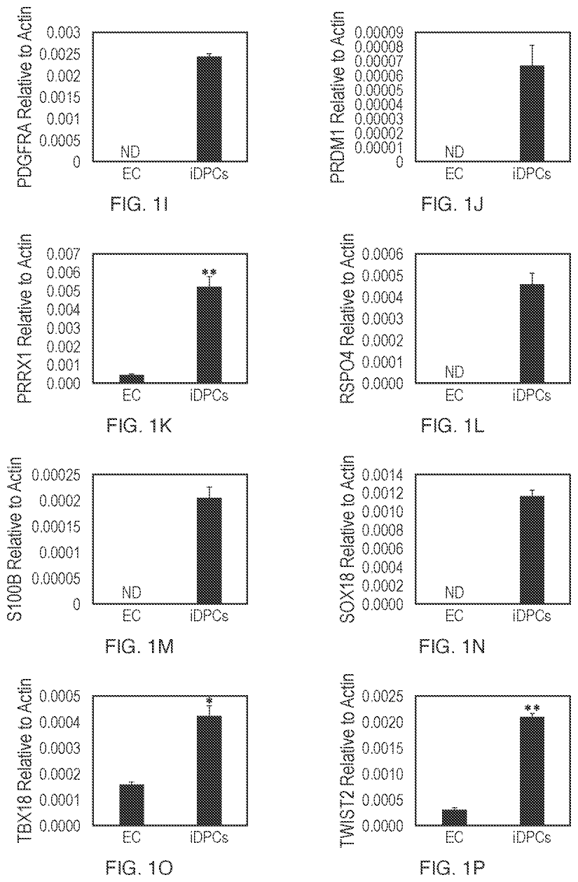

As shown in FIG. 1A-P, transduced cells show increased expression of the dermal papilla cell marker genes ALPL (FIG. 1A), EBF1 (FIG. 1B), ETV1 (FIG. 1C), FGF7 (FIG. 1D), FOXD1 (FIG. 1E), GLIS1 (FIG. 1F), GREM2 (FIG. 1G), ITGA9 (FIG. 1H), PDGFRA (FIG. 1I), PRDM1 (FIG. 1J), PRRX1 (FIG. 1K), RSPO4 (FIG. 1L), S100B (FIG. 1M), SOX18 (FIG. 1N), TBX18 (FIG. 1O), and TWIST2 (FIG. 1P) compared to ECs, indicating their transdifferentiation to iDPCs. Crossover threshold (CT) cutoff set to 30 cycles. ND represents values that were Not Detected or detected above the CT cutoff of 30 cycles. *P<0.005, **P<0.0005 for one-tailed T-test.

Alkaline phosphatase staining was used to verify protein-level expression of alkaline phosphatase in transduced cells. For alkaline phosphatase staining, cell cultures were fixed with 4% paraformaldehyde in phosphate buffered saline (PBS) for 2 minutes at room temperature and washed 3 times in PBS with 0.1% Triton-X 100. Alkaline phosphatase staining was performed by mixing reagents A and B from the Abcam Alkaline Phosphatase Staining Kit (Red) (ab242286) in a 1:1 v:v ratio and applying it to the fixed cells for 15 minutes at room temperature for all samples except P0 samples which were stained for 5 minutes due to robust staining. Following staining, cells were washed 1 time with PBS and 1 time with H₂O before drying at room temperature overnight.

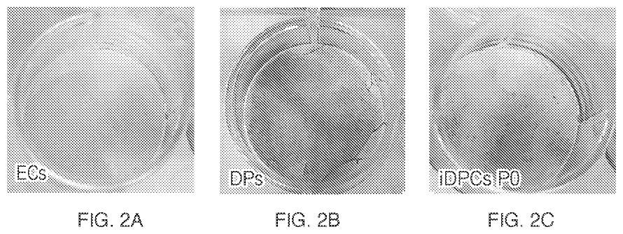

FIG. 2 shows whole-well images of alkaline phosphatase stainings of negative control ECs (FIG. 2A), DP positive control (FIG. 2B), and transduced cells (FIG. 2C). Alkaline phosphatase activity was not visible in negative control cells after 15 minutes of staining and prominent in positive control cells after 15 minutes of staining and in transduced cells after 5 minutes of staining, indicating that transduced cells were transdifferentiated into iDPCs.

### Example 2: Human iDPCS have the Capacity to Self-Renew

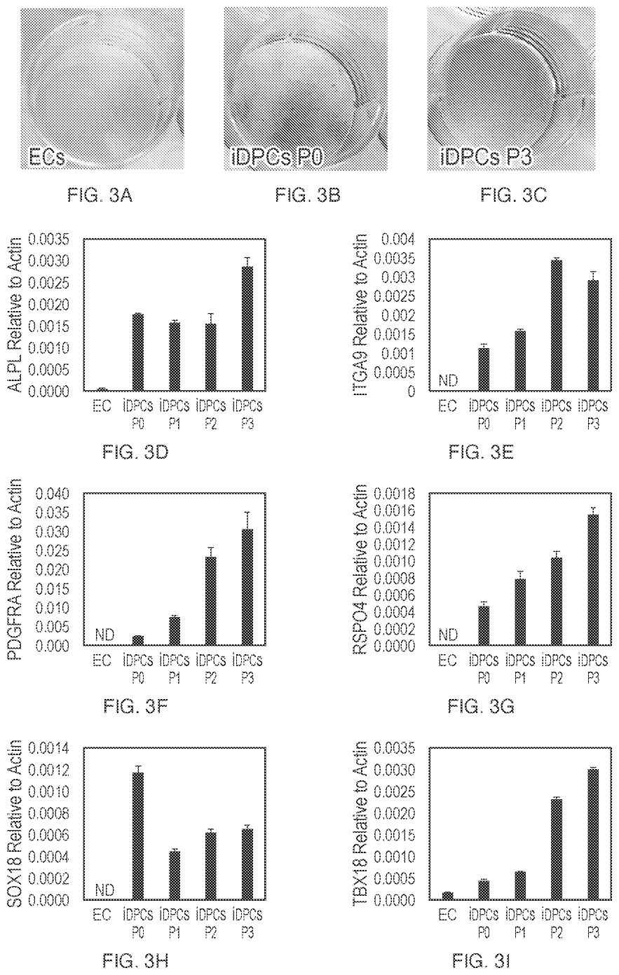

Maintenance of marker gene expression was used to analyze the ability of iDPCs to self-renew. The protocol from EXAMPLE 1 was used to generate iDPCs. Marker gene expression was assessed by alkaline phosphatase staining and qPCR analysis of mRNA isolated from iDPCs at passage (P) 0, 1, 2, and 3 and negative control human epithelial cells (EC) using the protocol from EXAMPLE 1. Positive alkaline phosphatase staining was maintained upon passage of iDPCs (FIGS. 3B and 3C) while staining was not detected in negative control ECs (FIG. 3A), indicating that iDPCs are capable of self-renewal. Primer nucleotide sequences used to detect actin, ALPL, ITGA9, PDGFRA, RSPO4, SOX18, and TBX18 are listed in TABLE 1 above. CT cutoff set to 30 cycles. ND represents values that were Not Detected or detected above the CT cutoff of 30 cycles. Expression values of ALPL (FIG. 3D), ITGA9 (FIG. 3E), PDGFRA (FIG. 3F), RSPO4 (FIG. 3G), SOX18 (FIG. 3H), and TBX18 (FIG. 3I) relative to actin are shown for iDPCs and negative control ECs. iDPCs maintain upregulated levels of ALPL, ITGA9, PDGFRA, RSPO4, SOX18, and TBX18 after multiple passages while these markers are not expressed at significant levels in negative control ECs, indicating that iDPCs are capable of self-renewal.

### Example 3: Isolation of Human iDPCS

Fluorescent Activated Cell Sorting (FACS) was used on iDPCs produced using the protocol described in EXAMPLE 1. Cultures of iDPCs were dissociated in TrypLE (ThermoFisher Scientific) for 5 minutes, washed in phosphate buffered saline (PBS), centrifuged at 300×g for 5 minutes and resuspended in staining solution (0.5% BSA in PBS). Cells were filtered through a 40 μm cell strainer and centrifuged for 5 minutes at 300× g prior to staining. For staining, cells were incubated for 30 minutes with recombinant anti-human ITGA9-FITC (Miltenyi Biotec) diluted 1:100 and rabbit anti-human PDGFR-Alexa Flour 647 (Abcam) diluted 1:1000 in staining solution on ice. Cells were then washed in staining solution, centrifuged at 300×g for 5 minutes, washed again with staining solution and centrifuged at 300×g for 5 minutes, then resuspended in staining solution supplemented with EDTA and Propidium Iodide (Invitrogen) according to the manufacturer's instructions. Stained samples were analyzed with an Attune NxT Flow Cytometer according to the manufacturer's instructions.

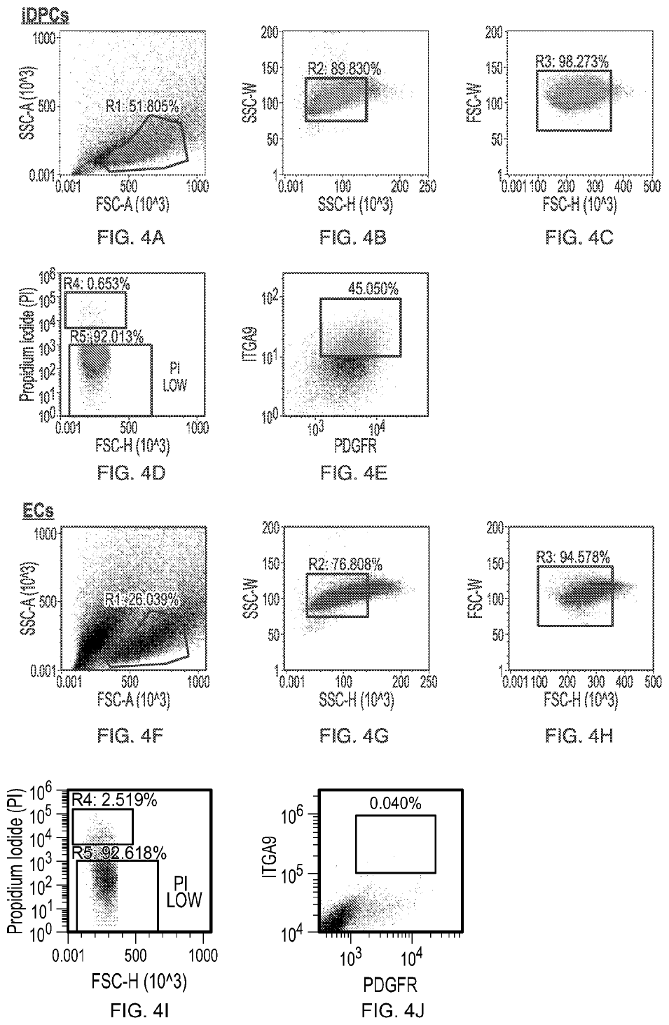

Flow cytometry analysis for iDPCs is shown in FIGS. 4A-4E while negative control ECs are shown in FIGS. 4F-4J. Gating parameters for cell size are shown in FIGS. 4A-4C and FIGS. 4F-4H. Dead cells were excluded by Propidium Iodide (PI) staining as shown in FIG. 4D and FIG. 41. ITGA9 and PDGFR analyses are shown in FIG. 4E and FIG. 4J. The boxes and accompanying numbers indicate the cell populations that are ITGA9/PDGFR double-positive, demonstrating the transdifferentiation of transduced cells into iDPCs. Furthermore, these iDPCs can be isolated and purified from a bulk population of cells by FACS based on ITGA9/PDGFR double positivity for further experimentation or usage.

### Example 4: Grafting of Human iDPCS onto Human Patients

iDPCs are used to grow hair in human patients for the treatment of human hair loss conditions such as androgenic alopecia, alopecia areata, telogen effluvium, trichotillomania, traction alopecia, tinea capitis, and cicatricial alopecia. The method described in EXAMPLE 1 is used to generate iDPCs from a patient's own cells, from an allogeneic donor, or from a cultured cell line. An appropriate number of cells, for example, but not limited to, 100,000, 1,000,000, or 10,000,000 iDPCs is placed on a suitable support, such as a plastic sheet or another suitable support comprised of Matrigel, a similar basement membrane extract, a synthetic hydrogel, or an extracellular membrane protein-based scaffold (e.g., collagen, fibronectin, gelatin, or laminin). iDPCs optionally are combined with human epidermal cells, cells functionally equivalent to epidermal cells, or dermal cells obtained from the patient, an allogeneic donor, or cell line. The support is grafted onto the patient at an appropriate site. The site of the graft is monitored in the short term (i.e., weeks to months) for the ability of the grafted iDPCs to produce hair, as well as in the long term (i.e., years to decades) for the ability of the grafted iDPCs to cause sustained hair growth. Adverse effects, such as skin irritation or other conditions that may arise from iDPC grafting, are monitored to assess the safety of iDPC grafting.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A composition comprising:
a population of non-dermal papilla (DPI) cells;
a population of induced dermal papilla cells (iDPCs) expressing one or more marker(s) selected from the group consisting of ALPL, EBF1, ETV1, FGF7, FOXD1, GLIS1, GREM2, ITGA9, PDGFRA, PRDM1, PRRX1, RSPO4, SIOOB, SOX18, TBX18, and TWIST2; and a dermal papilla cell reprogramming (DPCR) system comprising one or more dermal papilla cell reprogramming factor(s) selected from the group consisting of an EBF agent or a TWIST agent,
wherein the DPCR system causes trans differentiation of one or more non-DP cell into one or more iDPC.

2. The composition of claim 1, wherein the population of iDPCs have at least a two-fold increased expression of one or more marker(s), selected from the group consisting of ALPL, EBF1, ETV1, FGF7, FOXD1, GLIS1, GREM2, ITGA9, PDGFRA, PRDM1, PRRX1, RSPO4, SIOOB, SOX18, TBX18, and TWIST2, as compared to the population of non-dermal papilla cells.

3. The composition of claim 1, wherein the population of iDPCs have at least a fivefold increased expression of one or more marker(s) selected from the group consisting of ALPL, EBF1, ETV1, FGF7, FOXD1, GLIS1, GREM2, ITGA9, PDGFRA, PRDM1, PRRX1, RSPO4, SIOOB, SOX8, TBX18, and TWIST2, as compared to the population of non-dermal papilla cells.

4. The composition of claim 1, wherein the population of iDPCs have at least a ten-fold increased expression of one or more marker(s) as selected from the group consisting of ALPL, EBF, ETV1, FGF7, FOXD1, GLIS1, GREM2, ITGA9, PDGFRA, PRDM1, PRRX1, RSPO4, S10013, SOX18, TBX18, and TWIST2, as compared to the population of non-dermal papilla cells.

5. The composition of claim 1, wherein expression of the one or more marker(s) is determined by messenger RNA analysis using a method selected from the group consisting of quantitative polymerase chain reaction (PCR), reverse transcriptase PCR, RNA-Seq, or northern blot.

6. The composition of claim 1, wherein expression of the one or more marker(s) is determined by protein analysis using a method selected from the group consisting of flow cytometry, or western blot.

7. The composition of claim 1, wherein expression of the one or more marker(s) is maintained over at least one cell passage.

8. The composition of claim 1, wherein the EBF agent is an EBF polypeptide, or functional fragment thereof, fused to a permeant domain.

9. The composition of claim 1, wherein the TWIST agent is a TWIST polypeptide, or functional fragment thereof, fused to a permeant domain.

10. The composition of claim 8, wherein the permeant domain comprises an amino acid sequence selected from the group consisting of RQIKIWFQNRRMKWKK (SEQ ID NO: 1), RKKRRQRRR (amino acids 49-57 of HIV-1 tat; SEQ ID NO:2), TRQARRNRRRRWRERQR (amino acids 34-50 of HIV-1 rev; SEQ ID NO:3), RRRRRRRRR (R9; SEQ ID NO:4), and RRRRRRRR (R8; SEQ ID NO:5).

11. The composition of claim 1, wherein the EBF agent is a nucleic acid encoding an EFB polypeptide, or functional fragment thereof.

12. The composition of claim 1, wherein the TWIST agent is a nucleic acid encoding a TWIST polypeptide, or functional fragment thereof.

13. The composition of claim 11 or 12, wherein the nucleic acid comprises a tetracycline responsive promoter.

14. The composition of any one of claims 11 to 13, wherein the nucleic acid is within a capsid of a viral particle.

15. The composition of claim 1, wherein the population of non-DP cells are mammalian cells.

16. The composition of claim 15, wherein the population of non-DP cells are human or murine cells.

17. The composition of claim 15, wherein the population of non-DP cells are selected from the group consisting of dermal cells, epidermal cells, epithelial cells, adipocytes, and hematopoietic cells.

18. The composition of claim 17, wherein dermal cells are selected from the group consisting of fibroblasts, smooth muscle cells, and connective tissue cells.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. A composition comprising:
a population of non-dermal papilla (DP) cells;
a population of induced dermal papilla cells (iDPCs) expressing one or more marker(s) selected from the group consisting of ALPL, EBF1, ETV1, FGF7, FOXD1, GLIS1, GREM2, ITGA9, PDGFRA, PRDM1, PRRX1, RSPO4, SIOOB, SOX18, TBX18, and TWIST2; and a dermal papilla cell reprogramming (DPCR) system comprising one or more dermal papilla cell reprogramming factor(s) selected from the group consisting of an EBF agent,
wherein the DPCR system causes trans differentiation of one or more non-DP cell into one or more iDPC;
wherein the population of iDPCs have at least a two-fold increased expression of one or more marker(s), selected from the group consisting of ALPL, EBF1, ETV1, FGF7, FOXD1, GLIS1, GREM2, ITGA9, PDGFRA, PRDM1, PRRX1, RSPO4, SIOOB, SOX18, TBX18, and TWIST2, as compared to the population of non-dermal papilla cells; and
wherein the EBF agent is an EBF polypeptide, or functional fragment thereof, fused to a permeant domain, wherein the permeant domain comprises an amino acid sequence selected from the group consisting of: RQIKIWFQNRRMKWKK (SEQ ID NO:1), RKKRRQRRR (amino acids 49-57 of HIV-1 tat; SEQ ID NO:2), TRQARRNRRRRWRERQR (amino acids 34-50 of HIV-1 rev; SEQ ID NO:3), RRRRRRRRR (R9; SEQ ID NO:4), and RRRRRRR (R8; SEQ ID NO:5).

85. A composition comprising:
a population of non-dermal papilla (DP) cells;
a population of induced dermal papilla cells (iDPCs) expressing one or more marker(s) selected from the group consisting of ALPL, EBF1, ETV1, FGF7, FOXD1, GLIS1, GREM2, ITGA9, PDGFRA, PRDM1, PRRX1, RSPO4, SIOOB, SOX18, TBX18, and TWIST2; and a dermal papilla cell reprogramming (DPCR) system comprising one or more dermal papilla cell reprogramming factor(s) selected from the group consisting of a TWIST agent,
wherein the DPCR system causes trans differentiation of one or more non-DP cell into one or more iDPC;
wherein the population of iDPCs have at least a two-fold increased expression of one or more marker(s), selected from the group consisting of ALPL, EBF1, ETV1, FGF7, FOXD1, GLIS1, GREM2, ITGA9, PDGFRA, PRDM1, PRRX1, RSPO4, SIOOB, SOX18, TBX18, and TWIST2, as compared to the population of non-dermal papilla cells; and
wherein the TWIST agent is a TWIST polypeptide, or functional fragment thereof, fused to a permeant domain, wherein the permeant domain comprises an amino acid sequence selected from the group consisting of: RQIKIWFQNRRMKWKK (SEQ ID NO:1), RKKRRQRRR (amino acids 49-57 of HIV-1 tat, SEQ ID NO:2), TRQARRNRRRRWRERQR (amino acids 34-50 of HIV-1 rev; SEQ ID NO:3), RRRRRRRRR (R9; SEQ ID NO:4), and RRRRRRRR (R8; SEQ ID NO:5).
